(19) United States
(12) Patent Application Publication
(10) Pub. No.: US 2019/0175441 A1
(43) Pub. Date: Jun. 13, 2019

(54) SYSTEMS AND METHODS FOR CONTROLLING SEXUAL STIMULATION DEVICES

(71) Applicant: Karen URBANI, York (CA)

(72) Inventor: Karen URBANI, York (CA)

(21) Appl. No.: 15/834,167

(22) Filed: Dec. 7, 2017

(51) Int. Cl. A61H 19/00; G16H 40/63

(52) U.S. Cl. CPC A61H 19/50; A61H 19/32; G16H 40/63; A61H 19/34; A61H 2201/5005; A61H 2201/5046; A61H 2201/5074; A61H 2201/501; A61H 2201/5097

(57) ABSTRACT

A controller for a sexual stimulation device is disclosed. The controller includes: an input device formed as an artificial anatomical part, the input device including a plurality of touch sensors disposed thereon, the plurality of touch sensors being configured to generate output signals responsive to touch inputs on an external surface of the input device; and an attachment member for removably coupling the input device to a mobile electronic device, the mobile electronic device having a display on a first side, wherein the attachment member supports the input device over the first side of the mobile electronic device when the input device is secured to the mobile electronic device via the attachment member.

### TECHNICAL FIELD

The present disclosure relates to sexual aids and, in particular, to systems and methods for remotely controlling sexual stimulation devices.

### BACKGROUND

Sexual stimulation devices are widely used, both for personal use and for engaging in a stimulation experience with one or more partners. For partners that are remotely located from each other, it can be difficult to engage in an interactive sexual stimulation experience that is convenient and immersive for both partners. Traditional methods of visual stimulation (e.g. images, video chats, etc.) are limited in effecting real-time, physical stimulation for remote partners.

### BRIEF DESCRIPTION OF DRAWINGS

Reference will now be made, by way of example, to the accompanying drawings which show example embodiments of the present application and in which:

FIGS. 1A and 1B show a front view and a rear view, respectively, of an example mobile electronic device.

FIG. 1C is a block diagram illustrating components of an example mobile electronic device.

FIG. 2A shows a side elevational view of an example controller for a sexual stimulation device, in accordance with example embodiments of the present disclosure.

FIG. 2B shows a front view of another example controller for a sexual stimulation device.

FIG. 2C shows a front view of the example controller of FIG. 2B attached to a mobile electronic device.

FIG. 2D shows a perspective view of an example stimulation device which may be used with the example controller of FIGS. 2A-2C.

FIGS. 3A and 3B show a front view and a side elevational view, respectively, of another example controller for a mobile electronic device.

FIG. 3C shows a perspective view of an example stimulation device for use with the example controller of FIG. 3A.

FIG. 4A shows a perspective view of another example controller for a mobile electronic device.

FIG. 4B shows a perspective view of an example stimulation device for use with the example controller of FIG. 4A.

FIG. 5 shows, in flowchart form, an example method of controlling a sexual stimulation device in accordance with example embodiments of the present disclosure.

Like reference numerals are used in the drawings to denote like elements and features.

### DETAILED DESCRIPTION OF EXAMPLE EMBODIMENTS

In one aspect, the present disclosure describes a controller for a sexual stimulation device. The controller includes: an input device formed as an artificial anatomical part, the input device including a plurality of touch sensors disposed thereon, the plurality of touch sensors being configured to generate output signals responsive to touch inputs on an external surface of the input device; and an attachment member for removably coupling the input device to a mobile electronic device, the mobile electronic device having a display on a first side, wherein the attachment member supports the input device over the first side of the mobile electronic device when the input device is secured to the mobile electronic device via the attachment member.

In another aspect, the present disclosure describes a sexual stimulation system. The system includes a sexual stimulation device and a controller communicably coupled to the sexual stimulation device. The controller includes: an input device formed as an artificial anatomical part, the input device including a plurality of touch sensors disposed thereon, the plurality of touch sensors being configured to generate output signals responsive to touch inputs on an external surface of the input device; and an attachment member for removably coupling the input device to a mobile electronic device, the mobile electronic device having a display on a first side, wherein the attachment member supports the input device over the first side of the mobile electronic device when the input device is secured to the mobile electronic device via the attachment member. The sexual stimulation device is configured to: receive control signals that are generated responsive to output signals from the plurality of touch sensors of the controller; and generate vibrations based on the received control signals.

In yet another aspect, the present disclosure describes a method of controlling a sexual stimulation device. The method is implemented by a processor of a mobile electronic device, the mobile electronic device having a display on a first side. The method includes: receiving, from a controller for a sexual stimulation device, touch sensor output data, where the controller includes: an input device formed as an artificial anatomical part, the input device including a plurality of touch sensors disposed thereon, wherein the touch sensor output data is generated by the plurality of touch sensors responsive to touch inputs on an external surface of the input device; and an attachment member for removably coupling the input device to the mobile electronic device, the attachment member being configured to support the input device over the first side of the mobile electronic device when the input device is secured to the mobile electronic device via the attachment member. The method further includes: generating control signals based on the touch sensor output data, and transmitting the generated control signals to the sexual stimulation device.

Other example embodiments of the present disclosure will be apparent to those of ordinary skill in the art from a review of the following detailed descriptions in conjunction with the drawings.

The present disclosure describes systems for remotely operating sexual stimulation devices. The devices described herein may be used to facilitate interactive sexual stimulation among users that are remotely located from each other. In particular, the present disclosure provides a controller for sexual stimulation devices that is suitable for use with mobile electronic devices. The controller can be attached to a mobile electronic device, such as a smartphone or a tablet, so that it may be operated while simultaneously viewing content presented on a display of the mobile electronic device. The controller includes an input device for receiving touch inputs applied by a user and outputting touch sensor data responsive to the applied inputs. The touch sensor data is, in turn, used to generate control signals for controlling one or more remotely located sexual stimulation devices. The mobility of the controller offers flexibility as to where and how a sexual stimulation interaction may be undertaken, and allows convenient integration of control of sexual stimulation devices into a mobile user experience.

### Example Mobile Electronic Device

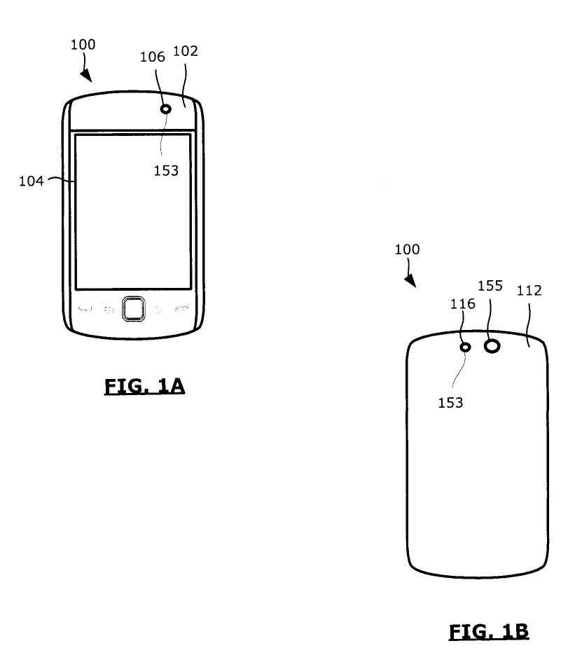

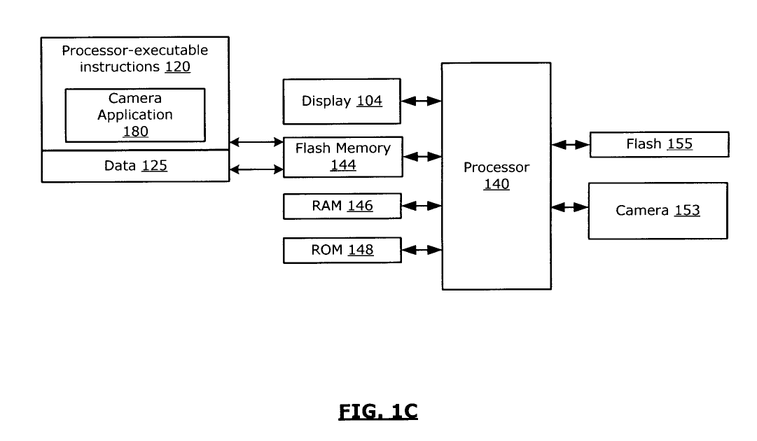

Reference is first made to FIGS. 1A and 1B, which show a front view and a rear view, respectively, of a mobile electronic device 100. The mobile electronic device 100 can, for example, be a mobile phone, portable computer, smartphone, tablet computer, wearable computer (e.g. smartwatch), a television, a digital camera, or a computer system. The mobile electronic device 100 may be of a form different from those specifically listed above.

FIG. 1A illustrates a front surface 102 of the mobile electronic device 100. The front surface 102 is a side of the mobile electronic device 100 which includes a display 104 that is configured to be viewed by a user of the mobile electronic device 100. The display 104 may be a touchscreen display which acts as both an input interface (i.e. touch-sensitive overlay) and an output interface (i.e. display). The display 104 may be constructed using a touch-sensitive input surface which is connected to an electronic controller and which overlays the display 104. For example, the touch-sensitive overlay and the electronic controller may provide a touch-sensitive input interface and a processor of the mobile electronic device 100 may interact with the touch-sensitive overlay via the electronic controller.

FIG. 1B illustrates a rear surface 112 of the mobile electronic device 100. The rear surface 112 is a side of the mobile electronic device 100 which does not include the main display 104. In the embodiment illustrated, the rear surface 112 is a side of the mobile electronic device 100 which is opposite to the front surface 102 (FIG. 1A) of the mobile electronic device 100. That is, the rear surface 112 may be substantially parallel to the front surface 102 of the mobile electronic device 100.

The mobile electronic device 100 includes one or more cameras 153. The cameras 153 are configured to generate camera data, such as images in the form of still photographs and/or motion video. The camera data may be captured in the form of an electronic signal which is produced by an image sensor associated with the camera 153. More particularly, the image sensor is configured to produce an electronic signal in dependence on received light. That is, the image sensor converts an optical image into an electronic signal, which may be output from the image sensor by way of one or more electrical connectors associated with the image sensor. The electronic signal represents electronic image data (which may also be referred to as camera data).

In the embodiment illustrated, the front surface 102 includes a front facing camera 106. A front facing camera is a camera which is located to obtain images of a subject near the front surface 102 of the mobile electronic device 100. That is, the front facing camera 106 may be generally located at or near the front surface 102 of the mobile electronic device 100. The front facing camera 106 may be located anywhere on the front surface 102; for example, the front facing camera 106 may be located above or below the display 104. In at least some embodiments, the front facing camera 106 may be provided in a central location relative to the display 104, to facilitate image acquisition of a face. The front facing camera 106 may be used, for example, to allow a user of the mobile electronic device 100 to engage in a video-based chat with a user of another mobile electronic device or to capture images of the user's own face. In some embodiments, the front facing camera 106 is mounted internally within a housing of the mobile electronic device 100, beneath a region of the front surface 102 which transmits light. For example, the front facing camera 106 may be mounted beneath a clear portion of the housing of the mobile electronic device 100 which allows light to be transmitted to the internally mounted front facing camera 106.

Similarly, the rear surface 112 includes a rear facing camera 116. A rear facing camera 116 is a camera 153 which is located to obtain images of a subject near the rear surface 112 of the mobile electronic device. That is, the rear facing camera may be located on or near the rear surface 112 of the mobile electronic device 100. For example, the rear facing camera 116 may be mounted internally within a housing of the mobile electronic device 100 beneath a region of the rear surface 112 which transmits light.

The mobile electronic device 100 may also have additional camera hardware to complement the cameras 153. For example, in the embodiment illustrated in FIG. 1B, the mobile electronic device 100 includes a flash 155. The flash may, in some embodiments, be a light emitting diode (LED) flash. In FIG. 1B, the flash 155 is located to emit light at the rear surface 112 of the mobile electronic device 100. That is, the flash 155 is a rear facing flash in the illustrated embodiment.

Reference is now made to FIG. 1C which illustrates a block diagram of components of the mobile electronic device 100. Some of the subsystems shown in FIG. 1C perform communication-related functions, whereas other subsystems may provide “resident” or on-device functions. The mobile electronic device 100 includes a controller including at least one processor 140 (such as a microprocessor) which controls the overall operation of the mobile electronic device 100. The processor 140 may be communicatively connected with device subsystems such as one or more output interfaces (such as a display 104, a flash 155 and/or a speaker), one or more input interfaces (such as a camera 153, control buttons, a touch-sensitive overlay associated with a touchscreen display, and/or other input interfaces), memory (such as flash memory 144, random access memory (RAM) 146, read only memory (ROM) 148, etc.), data port 170, power port 175, short-range communication subsystem 180, and other device subsystems generally designated as 190.

The data port 170 may include a serial port, a parallel port, and/or another type of connection port. In some implementations, the data port 170 is a Universal Serial Bus (USB) port that includes data lines for data transfer and a supply line that can provide a charging current to charge a battery of the mobile electronic device 100. The mobile electronic device 100 may be manually synchronized with a host system, for example, by connecting the mobile electronic device 100 through the data port 170 (e.g. in an interface cradle and/or another type of wired connection) that couples the mobile electronic device 100 to a data port of a computer system or other device. The data port 170 may also be used to enable a user to set preferences through an external device or software application, or to download other programs for installation.

The short-range communication subsystem 180 provides for communication between the mobile electronic device 100 and different systems and/or devices, which need not necessarily be similar devices. For example, the subsystem 180 may include an infrared device and associated circuits and components, or a wireless bus protocol compliant communication mechanism such as a Bluetooth® communication module to provide for communication with similarly-enabled systems and devices.

The mobile electronic device 100 stores data 125 in an erasable persistent memory, which, in one example embodiment, is the flash memory 144. In some embodiments, the data 125 includes images generated by the camera 153. The processor 140 operates under stored program control and executes processor-executable instructions 120 stored in memory such as persistent memory, for example, in the flash memory 144.

The processor-executable instructions 120 or parts thereof may be temporarily loaded into volatile memory such as the RAM 146. The RAM 146 may be used for storing runtime data variables and other types of data or information, as will be apparent. Although specific functions are described for various types of memory, this is merely one example. It will be appreciated that a different assignment of functions to types of memory could also be used.

While the memory which stores the processor-executable instructions 120 has been illustrated using a block that is separate from the processor 140, in practice, the memory storing the processor-executable instructions 120 may be provided on-board the processor 140. That is, the processor 140 may include internal memory and the processor-executable instructions may be provided on the internal memory.

The mobile electronic device 100 may include other components apart from those illustrated in FIG. 1C. By way of example, the mobile electronic device 100 may include or be connected to a power source, such as a battery, which provides power to various components of the electronic device.

### Controller for a Sexual Stimulation Device

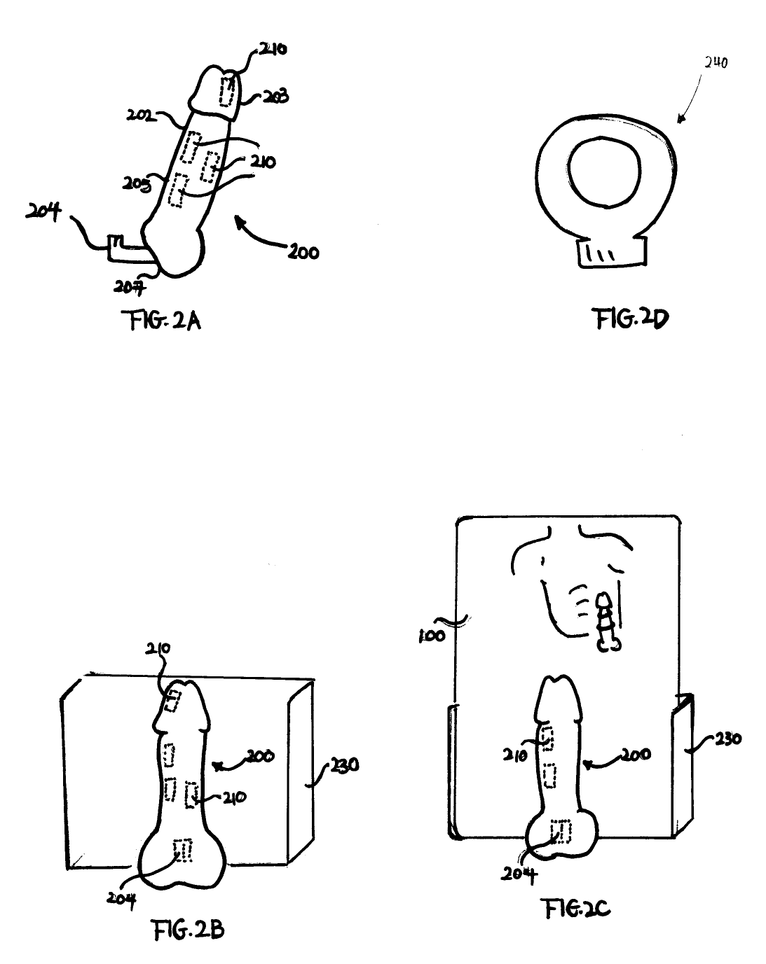

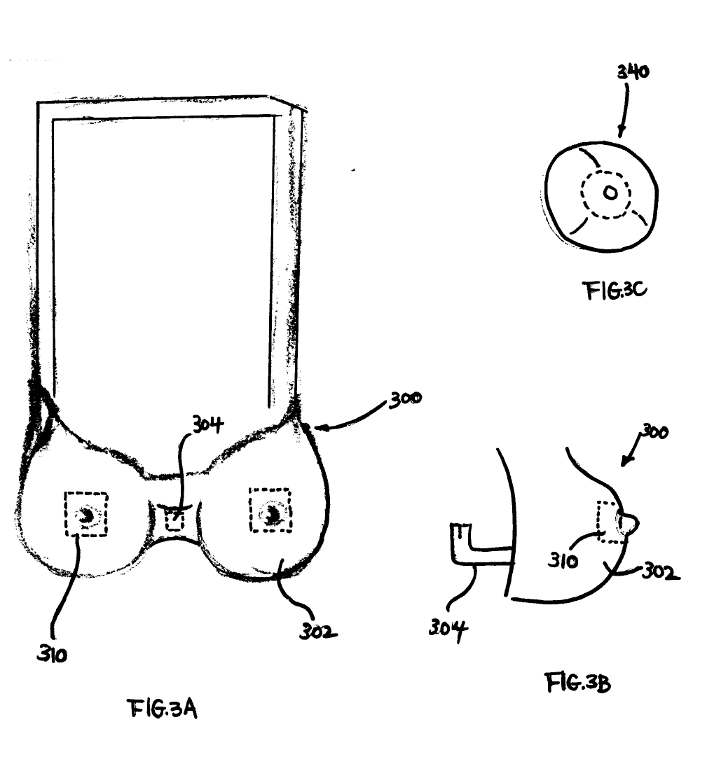

Reference is now made to FIGS. 2A-2C, which show example controllers for sexual stimulation devices. FIG. 2A shows an example controller 200. The controller 200 may be operated by a first user to control a sexual stimulation device associated with a second user, who may be remotely located from the first user. The controller 200 may also be operated by a user for self-pleasure, by connecting the controller 200 to a sexual stimulation device worn by or applied on the user. In some embodiments, the controller 200 may be used to control two or more different sexual stimulation devices associated with, for example, multiple different participants.

The controller 200 is suitable for use with a mobile electronic device 100, such as a mobile phone or a tablet. In particular, the controller 200 may be physically attached to the mobile electronic device 100 and configured to communicate, via either a wireless or wired connection, directly with the mobile electronic device 100. Accordingly, the controller 200 may be provided in various different sizes to accommodate use with different types of mobile electronic devices. For example, the dimensions of a controller 200 that is designed for use with a mobile phone may be smaller than the dimensions of a controller 200 that is designed for use with a tablet.

The controller 200 includes an input device 202. The input device 202 is formed as an artificial anatomical part. More specifically, the input device 202 is shaped to resemble, in appearance, one or more human anatomical parts. For example, at least part of the input device 202 may be formed in the shape of as a hand, a foot, a thigh, one or more parts of male (e.g. penis, scrotum) or female (e.g. clitoris, vulva) genitals, female breasts, shoulder, buttocks, or anus. In at least some embodiments, the input device 202 may comprise a sex toy or any object or device that is traditionally used to facilitate human sexual pleasure. For example, the input device 202 may integrate a dildo, an artificial vagina, or silicone breasts. The input device 202 may be constructed of one or more elastic materials, such as electrically conductive silicone (e.g. silicone rubber, foamed silicone, etc.), glass, plastic, vinyl, latex, and/or rubber (e.g. graphene rubber).

The input device 202 includes a plurality of touch sensors 210 that are disposed in the input device 202. The touch sensors 210 are used to detect touch inputs (i.e. single- or multi-touch inputs) that are applied to the input device 202. The touch sensors 210 may, for example, include resistive or capacitive sensors that are embedded at select locations on the input device 202. For example, the touch sensors 210 may be located at or near the external surface of the input device 202. Specifically, the touch sensors 210 may be located at select positions on the input device 202 that correspond to predetermined locations of an artificial anatomical part. In FIG. 2A, the input device 202 is formed as a phallic device having a plurality of internally positioned touch sensors 210. The input device 202 includes a head portion 203 and a shaft portion 205 adjacent to the head portion 203, and at least one touch sensor 210 may be embedded at one or both of the head portion 203 and the shaft portion 205. Alternatively, in some embodiments, the touch sensing area of the input device 202 may encompass the entire external surface of the input device 202. That is, rather than having touch sensors 210 located at predetermined locations, the input device 202 may include a flexible, touch-sensitive overlay which covers all or substantially all of the external surface of its external surface.

The touch sensors 210 may be of known types, such as contact sensors, pressure sensors, bend sensors, stretch sensors, compression sensors, or other touch-based sensors. In particular, the touch sensors 210 may be used to determine attributes of a touch event, such as the position and/or applied pressure of a touch input. As the touch sensors 210 are deployed at or under the external surface of the input device 202, gestures such as rubbing, stroking, grasping, stretching, squeezing, and bending parts of the input device 202 may activate the embedded touch sensors 210. The input device 202 may, in some embodiments, include additional sensors, such as a gyroscope, an accelerometer, a moisture sensor, thermometer, and/or a galvanic skin response sensor.

The touch sensors 210 are configured to generate output signals in response to detecting touch inputs on an external surface of the input device 202. The controller 200 may include a processor or a control circuit (not shown) that is connected to the touch sensors 210 and that receives signals outputted by the touch sensors 210. The processor/control circuit may process the output signals or transmit the output signal data to the mobile electronic device 100 for processing, in order to determine the position and/or applied pressure associated with detected touch inputs.

The controller 200 also includes an attachment member 204 for removably coupling the input device 202 to the mobile electronic device 100. In FIG. 2A, the attachment member 204 includes a bus connector, which may be a data and/or power connector, such as a Universal Serial Bus (USB) connector. In particular, a USB plug of the attachment member 204 may matingly engage a receptacle of a port on the mobile electronic device 100 to attach the input device 202 to the mobile electronic device 100. The connector extends from the body of the input device 202 in a direction that is opposite to the general orientation of the input device 202 when faced toward the user, and is of sufficient length to enable a mating connection to the port of the mobile electronic device 100. For example, as illustrated in FIG. 2A, the connector extends rearward away from the input device 202 such that when the controller 200 is attached to the mobile electronic device 100, the rear end 207 of the input device 202 is adjacent to the mobile electronic device 100. Modern mobile electronic devices generally include a data/power port on a bottom surface. Accordingly, the input device 202 may be attached adjacent to the bottom surface of the mobile electronic device 100 by inserting the attachment member/connector 204 into the port of the mobile electronic device 100. Since the location of the port may vary device to device, the position of the attachment member/connector 204 may be adjustable (e.g. horizontally adjustable) to facilitate proper engagement (i.e. alignment) between the connector and the port.

The connector also allows output signals from the touch sensors 210 to be transmitted to the mobile electronic device 100. In particular, the connector serves to establish a data connection between the input device 202 and the mobile electronic device 100, allowing for transfer of sensor data output by the touch sensors 210.

As shown in FIG. 2C, when the input device 202 is secured to the mobile electronic device 100, the input device 202 is supported, at least in part by the attachment member 204, over a side of the mobile electronic device 100 that includes a display. This arrangement of the input device 202 relative to the mobile electronic device 100 and the display thereon allows a user of the controller 200 to be able to view content, such as images or videos, being presented on the display. For example, the user may engage in a video chat with a partner using a sexual stimulation device which the user is controlling via the controller 200.

In some embodiments, the attachment member 204 may include additional components which facilitate secure physical connection between the input device 202 and the mobile electronic device 100. For example, the attachment member 204 may include one or more walls 230 which define a cradle for receiving the mobile electronic device 100. The walls 230 may include, at least, lateral sidewalls 231 and 232 which define an interior space therebetween, generally corresponding to a width of the mobile electronic device 100. The walls 230 may be flexible and biased inwards such that the mobile electronic device 100 may form a press fit with the walls 230 when the mobile electronic device 100 is secured using the attachment member 204.

FIG. 2D shows an example sexual stimulation device 240. In FIG. 2D, the device 240 is a vibration ring which may be suitable for use with the controller 200. A vibrating ring can be slipped around the shaft of a user's penis and produce small vibrations to provide stimulation for the user. The device 240 includes a communication subsystem which allows for remote communication with the controller 200, either via short-range wireless communication (e.g. Bluetooth®) or a wireless network (e.g. WWAN, WLAN, etc.). As will be explained in greater detail below, one or more vibrating rings may be worn by a second user and controlled by a remotely located first user using the controller 200. Other stimulation devices, such as variations of penis rings (e.g. erection rings, etc.) and mechanical masturbators, may be adapted to be used in conjunction with the controller 200. In particular, the touch inputs (e.g. rubbing, stroking, etc.) applied by the first user may be used to generate control signals for controlling the vibrations produced by the vibrating ring(s) worn by the second user. More generally, the device 240 is remotely controlled, in real-time, based on the control signals generated from the touch inputs detected on the input device 202 of controller 200. The device 240 receives control signals transmitted from the mobile electronic device 100 that is coupled to the controller 200, and generates vibrations based on the received control signals.

FIG. 3A shows another example controller 300 for a sexual stimulation device 340. The controller 300 includes an input device 302 which is formed as a pair of artificial breasts having a plurality of internally positioned touch sensors 310. In particular, the input device 302 includes a nipple portion 306, and one or more of the touch sensors 310 are located at the nipple portion 306. The touch sensors 310 may also be located at other positions on the input device 302. The controller 300 also includes an attachment member 304, which may include a USB plug projecting away from the input device 302. The connector enables data transfer between the input device 302 and the mobile electronic device 100. The attachment member 304 may also include walls 330 which define a cradle for receiving the mobile electronic device 100. The attachment mechanism of the controller 300 allows at least a part of the display of the mobile electronic device 100 to be visible when the input device 302 is attached and secured to the mobile electronic device 100. In the example of FIG. 3C, the sexual stimulation device 340 which may be cooperatively used with the controller 300 is an electronic breast stimulator, such as a silicone breast stimulating tickler/cup, or bra vibrator. The device 340 is communicably coupled to the mobile electronic device 100 and is configured to receive control signals to trigger vibrations, based on touch inputs detected on the input device 302.

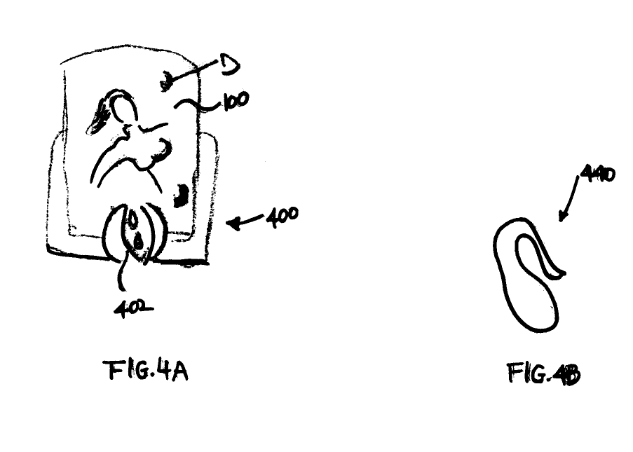

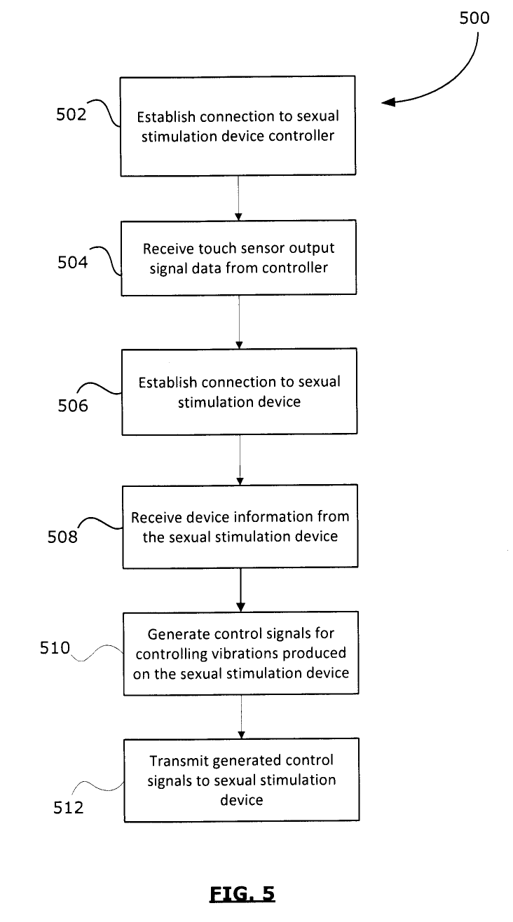

FIG. 4A shows another example controller 400 for a sexual stimulation device 440. The controller 400 includes an input device 402 which is formed as an artificial vagina. The controller 400 also includes one or more touch sensors 410 which may, for example, be located at or around a clitoris portion (corresponding to an actual location of the female clitoris) or a G-spot portion of the device 440. The controller 400 also includes an attachment member 404, which may include a USB plug projecting away from the input device 402. The connector enables data transfer between the input device 402 and the mobile electronic device 100. The attachment member 404 may also include walls 430 which define a cradle for receiving the mobile electronic device 100. In the example of FIG. 4B, the sexual stimulation device 440 which may be cooperatively used with the controller 400 is a vaginal or clitoral vibrator. The device 440 is communicably coupled to the mobile electronic device 100 and is configured to receive control signals to trigger vibrations, based on touch inputs detected on the input device 402.

### Method for Controlling a Sexual Stimulation Device

Reference is now made to FIG. 5 which shows, in flowchart form, an example method 500 for controlling a sexual stimulation device, using a controller such as example controller 200, 300, or 400 described above. The method 500 may be implemented by a processor of a mobile electronic device, such as mobile electronic device 100 of FIGS. 1A-1C. In particular, the method 500 describes operations of a mobile electronic device which is paired/connected to a controller for a sexual stimulation device. In some embodiments, software applications, such as a mobile application, installed on the mobile electronic device may be designed to perform some or all of the steps of method 500.

In step 502, a connection is established between a first mobile electronic device and the controller for the sexual stimulation device. A prompt for a connection may be triggered if, for example, a connector associated with the controller engages a port on the first mobile electronic device. For example, if a USB plug on the controller matingly connects to a data/power port of the first mobile electronic device, a request for connection may be made on the first mobile electronic device. The pairing of the first mobile electronic device and the controller may alternatively be made when a request to connect is made, via a wireless communication (e.g. a message transmitted using Bluetooth® or NFC technologies), from the controller to the first mobile electronic device, or when the first mobile electronic device discovers/detects a nearby controller device using, for example, Wi-Fi or Bluetooth®.

After a data connection is established between the first mobile electronic device and the controller, output sensor data from the touch sensors associated with the controller is received at the first mobile electronic device, in step 504. The sensor data may be received via a USB connection established using the connector on the controller or using wireless communication between the first mobile electronic device and the input device of the controller. The transmission of the sensor data may be initiated as soon as touch inputs are detected on the input device of the controller after a connection between the first mobile electronic device and controller is established. Alternatively, a software application/module on the first mobile electronic device may regulate the transmission and receipt of sensor data from the controller. For example, sensor data from the touch sensors of the controller may only be accepted at the first mobile electronic device after an express instruction is provided via a mobile application on the first mobile electronic device to request transmission from the controller.

In step 506, a wireless connection is established between the first mobile electronic device and the remotely located sexual stimulation device that is controlled using the controller, and device information for the sexual stimulation device is received in step 508. For example, device identifying information, such as type, size, and feature settings may be received at the first mobile electronic device. Other information, such as user preference and usage history, may also be received from the sexual stimulation device.

In at least some embodiments, the sexual stimulation device may connect to the first mobile electronic device via a second mobile electronic device. That is, the sexual stimulation device may be communicably coupled to a second mobile electronic device which, in turn, is able to establish a connection with the first mobile electronic device. For example, the first and second mobile electronic devices may, respectively, be operated by participants of a sexual stimulation interaction. The sexual stimulation device may connect to the second mobile electronic device via a wired (e.g. via a USB connection) or wireless (e.g. Bluetooth®, NFC, Wi-Fi, etc.) connection, and may exchange data over said connection.

In step 510, control signals for controlling vibrations to be produced on the sexual stimulation device are generated at the mobile electronic device. The control signals include, for example, instructions which indicate at least one of location, timing, frequency, and intensity of vibrations to be generated on the sexual stimulation device. If multiple sexual stimulation devices are controlled simultaneously by the controller (e.g. two or more vibrations rings worn by the user), the instructions may also indicate which of the sexual stimulation devices is to produce vibrations and at what time such vibrations are to be produced.

In at least some embodiments, the vibration effects produced on the sexual stimulation device(s) depend on the attributes of the user touch inputs on the input device of the controller. For example, a user's stroking of the input device may be interpreted as a moving touch input, and accordingly, the position of the touch input may determine which of multiple stimulation devices or which parts of a single stimulation device will receive a vibrating force. As another example, the force that is applied by a grasping of at least part of the input device (such as a phallic device or artificial breasts), measured for example by a pressure sensor, may determine the frequency or intensity of vibration (e.g. by varying voltage applied to one or more vibrations motors) applied to the sexual stimulation device. Sensor data from other types of sensors, such as a bend sensor or accelerometer, may affect the properties of the vibrations produced on the sexual stimulation device.

In step 512, the generated control signals are transmitted to the sexual stimulation device. In some embodiments, the control signals are transmitted to a second mobile electronic device that is communicably connected to the sexual stimulation device. For example, a remote user may connect the sexual stimulation device to the second electronic device (e.g. controlled by a software application/module on the second electronic device). The second electronic device may be used to control or adjust settings of the sexual stimulation device, record a live video of the stimulation interaction using a camera of the second electronic device, and communicate with the controller and the associated mobile electronic device. In particular, the mobile electronic device may be configured to receive, from the second mobile electronic device, video data acquired by a camera associated with the second mobile electronic device while the controller is used to simultaneously control the operation of the sexual stimulation device, and present the received video data on the display. The video data may, for example, be a live stream video of the user's partner as they interact with the sexual stimulation device. This allows the user to engage in an immersive experience by remotely controlling the sexual stimulation device while watching a live video of the effects of the user's control input.

The various embodiments presented above are merely examples and are in no way meant to limit the scope of this application. Variations of the innovations described herein will be apparent to persons of ordinary skill in the art, such variations being within the intended scope of the present application. In particular, features from one or more of the above-described example embodiments may be selected to create alternative example embodiments including a sub-combination of features which may not be explicitly described above. In addition, features from one or more of the above-described example embodiments may be selected and combined to create alternative example embodiments including a combination of features which may not be explicitly described above. Features suitable for such combinations and sub-combinations would be readily apparent to persons skilled in the art upon review of the present application as a whole. The subject matter described herein and in the recited claims intends to cover and embrace all suitable changes in technology.

1. A sexual stimulation system, comprising:
a controller for a sexual stimulation device, the controller including:
an input device formed as an artificial anatomical part, the input device including a plurality of touch sensors disposed thereon, the plurality of touch sensors being configured to generate output signals responsive to touch inputs on an external surface of the input device; and
an attachment member for removably coupling the input device to a mobile electronic device, the mobile electronic device having a display on a first side, wherein the attachment member supports the input device over the first side of the mobile electronic device when the input device is secured to the mobile electronic device via the attachment member, and
a sexual stimulation device communicably connected to the controller, the stimulation device being configured to:
receive control signals that are generated responsive to output signals from the plurality of touch sensors of the controller; and
generate vibrations based on the received control signals.

2. The system of claim 1, wherein the mobile electronic device includes a processor that is configured to:
receive signal data outputted by the plurality of touch sensors;
generate control signals based on the received output signal data; and
transmit the generated control signals to the sexual stimulation device.

3. The system of claim 2, wherein the attachment member includes a connector that is communicably connected to the input device and is configured to directly engage a port on the mobile electronic device.

4. The system of claim 3, wherein the output signal data is transmitted to the processor via the port on the mobile electronic device.

5. The system of claim 1, wherein the stimulation device comprises a vibrating ring.

6. The system of claim 1, wherein the stimulation device comprises an electronic breast stimulator.

7. A controller for a sexual stimulation device, the controller comprising:
an input device formed as an artificial anatomical part, the input device including a plurality of touch sensors disposed thereon, the plurality of touch sensors being configured to generate output signals responsive to touch inputs on an external surface of the input device; and
an attachment member for removably coupling the input device to a mobile electronic device, the mobile electronic device having a display on a first side, wherein the attachment member supports the input device over the first side of the mobile electronic device when the input device is secured to the mobile electronic device via the attachment member.

8. The controller of claim 7, wherein the attachment member includes a connector that is communicably connected to the input device and is configured to directly engage a port on the mobile electronic device.

9. The controller of claim 7, wherein the plurality of touch sensors includes at least one of a contact sensor and a pressure sensor.

10. The controller of claim 7, wherein the input device comprises a phallic device having a plurality of internally positioned touch sensors.

11. The controller of claim 10, wherein the phallic device includes a head portion and a shaft portion and wherein the plurality of touch sensors are located at one or both of the head portion and the shaft portion.

12. The controller of claim 7, wherein the input device is formed as a pair of artificial breasts having a plurality of internally positioned touch sensors.

13. The controller of claim 12, wherein each of the artificial breasts includes a nipple portion and wherein one or more of the touch sensors are located at the nipple portion.

14. The controller of claim 7, wherein the input device is formed as an artificial vagina.

15. A method, implemented by a processor of a mobile electronic device, for controlling a sexual stimulation device, the mobile electronic device having a display on a first side, the method comprising:
receiving, from a controller for a sexual stimulation device, touch sensor output data, the controller including:
an input device formed as an artificial anatomical part, the input device including a plurality of touch sensors disposed thereon, wherein the touch sensor output data is generated by the plurality of touch sensors responsive to touch inputs on an external surface of the input device; and
an attachment member for removably coupling the input device to the mobile electronic device, the attachment member being configured to support the input device over the first side of the mobile electronic device when the input device is secured to the mobile electronic device via the attachment member;
generating control signals based on the touch sensor output data; and
transmitting, to the sexual stimulation device, the generated control signals.

16. The method of claim 15, wherein the control signals comprise instructions to cause controlled generation of vibrations on the sexual stimulation device.

17. The method of claim 16, wherein the instructions indicate at least one of location, timing, frequency, and intensity of vibrations to be generated on the sexual stimulation device.

18. The method of claim 15, wherein the control signals are generated based on at least one of location and pressure of touch inputs applied on the external surface of the input device.

19. The method of claim 15, wherein transmitting the generated control signals to the sexual stimulation device comprises transmitting the generated control signals to a second mobile electronic device that is communicably coupled to the sexual stimulation device.

20. The method of claim 19, further comprising:
receiving, from the second mobile electronic device, video data acquired by a camera associated with the second mobile electronic device while simultaneously transmitting the generated control signals to the sexual stimulation device; and
presenting the received video data on the display.
